(19) United States
(12) Patent Application Publication
(10) Pub. No.: US 2018/0014784 A1
(43) Pub. Date: Jan. 18, 2018

(54) SYSTEM AND METHOD FOR ELECTROPHYSIOLOGICAL MONITORING

(71) Applicant: New York University, NEW YORK, NY (US)

(72) Inventors: David J. Heeger, NEW YORK, NY (US); André A. Fenton, NEW YORK, NY (US)

(21) Appl. No.: 15/547,018

(22) PCT Filed: Feb. 1, 2016
(86) PCT No.: PCT/US16/15971

§ 371 Date: Jul. 27, 2017

Related U.S. Application Data
(60) Provisional application No. 62/110,118, filed on Jan. 30, 2015.

(51) Int. Cl. A61B 5/00; A61B 5/0496; A61B 5/0492; A61B 5/08; A61B 5/0408; A61B 5/0478

(52) U.S. Cl. CPC A61B 5/6843; A61B 5/6804; A61B 5/04087; A61B 5/0478; A61B 5/0492; A61B 5/6892; A61B 5/0006; A61B 5/0809; A61B 5/0496; A61B 2503/04; A61B 2560/0214; A61B 2562/0209; A61B 2562/222

(57) ABSTRACT

A system and method for the acquisition and analysis of physiological data, such as for example, electrophysiological data including but not limited to EEG, EKG, EMG EOG, and biomechanical data relating to breathing and/or respiration to provide further insight to the person's health and/or behavior. A plurality of self-contained sensors having an electrically-conducting interface, an amplifier, an analog to digital converter, and a wireless transceiver may be used. Each of the self-contained sensors may be about the size of a watch battery. A sensor array embedded within a conductive fabric may also be used to detect physiological data. The conductive fabric may be included a plurality of conduct nodes rising out of a surface of the conductive fabric or may be a quilted meshwork having a plurality of sections, each section forming a conductive surface for detecting physiological data.

### PRIORITY CLAIM

This application claims priority to U.S. Provisional Application Ser. No. 62/110,118 filed Jan. 30, 2015, the entire contents of which is hereby incorporated by reference herein.

### FIELD OF INVENTION

The present invention relates generally to systems and methods for the acquisition and analysis of physiological data, such as for example, electrophysiological data and/or data acquired by other types of physiological measurements.

BACKGROUND

There has been an increasing interest in measuring and analyzing different types of physiological data in a person to better understand different biometric factors within the person. One particular movement for collecting and analyzing various types of physiological data is referred to as quantified self. Specifically, quantified self (also called self-tracking, auto-analytics, body hacking, self-quantifying, self-surveillance, life logging, etc.) is a movement to incorporate technology into a person's daily life for acquiring, analyzing, and comparing data on aspects of the person's daily life, to improve the person's life. Such self-monitoring may include data collected manually by the person or by a wearable sensor that is worn by the person for a period of time, e.g., throughout the day, that measures and collects physiological data from the person. The data collected may be analyzed to provide further insight to the person's behavior or health.

Although there has been an increasing level of interest in obtaining and analyzing different types of physiological data, the technology currently available is still very limiting. All too often, the person is required to provide data by manual input, which is often cumbersome, tedious, and introduces potential errors to the data. In other cases, the technology utilized, for example, a pedometer or a fitness tracker, is limited in its ability to collect physiological data. Other forms of physiological data may include different types of electrophysiological data, such as, for example, data obtained by electroencephalography (EEG), electrocardiogram (EKG or ECG), electrooculogram (EOG), electromyogram (EMG), each of which provides an example of a technique, method, and apparatus for measuring an electrophysiological signal. These electrophysiological signals have been previously measured in medical applications. For example, EEG has previously been used to diagnose epilepsy and sleep disorders. However, recording of these electrophysiological signals typically involves equipment that is cumbersome to operate, requires in-person visits to a medical facility, and/or is costly to administer. For example, the recording equipment may be attached by wires to large and uncomfortable electrodes in contact with the person. The equipment may also require installation and operation by a trained technician. Further, the recording equipment may include electrodes connected by wires to a separate electronic device for performing analysis. In addition, an electrically-conducting gel is typically used to make an electrical contact between the electrodes (typically metal pads) and the person's skin, which is messy and cannot be used easily while the person is moving about in their day-to-day activities.

Therefore, there is a continuing need in the art for improved systems and methods for measuring and analyzing different types of physiological data, particularly electrophysiological data, to provide further insight to the person's health and/or behavior.

### SUMMARY OF THE INVENTION

In accordance with the foregoing objectives and others, one embodiment of the present invention provides a sensor for detecting physiological signals in a person. The sensor includes an electrically-conducting interface configured to be placed in contact with the person to detect an electrophysiological signal or biomechanical data from the person. The sensor also includes a wireless transceiver configured to transmit data corresponding to the electrophysiological signal to a receiver external to the sensor. The sensor does not include a wire extending therefrom connecting the sensor to any other electric device.

In another aspect, a sensor array for detecting physiological signals in a person is provided. The sensor array comprises a plurality of conductive nodes each configured to be placed in contact with a person to detect an electrophysiological signal or biomechanical data from the person. The sensor array also comprises a conductive fabric comprising a plurality of electrically conducting threads or wires embedded therein. The plurality of conductive nodes may be integrated within the conductive fabric. In addition, each of the conductive nodes may be electrically isolated from other conductive nodes.

In a further aspect, a physiological monitoring system is provided. The system comprises a plurality of sensors each configured to be placed in contact with a person to detect an electrophysiological signal or biomechanical data from the person. Each of the plurality of sensors are independently operated such that the sensors do not share a common ground and the sensors do not share a common time registration. The system also comprises a receiver configured to wirelessly receive data corresponding to the electrophysiological signal or biomechanical data detected by each of the plurality of sensors. The system further comprises a processing arrangement.

These and other aspects of the invention will become apparent to those skilled in the art after a reading of the following detailed description of the invention, including the figures and appended claims.

### BRIEF DESCRIPTION OF THE FIGURES

FIG. 1 shows an exemplary embodiment of a sensor for detecting various different types of electrophysiological data or biomechanical data from a person according to the present invention.

FIG. 2 shows an alternative embodiment of a sensor for detecting various different types of electrophysiological data or biomechanical data from a person according to the present invention.

FIG. 3 shows an exemplary embodiment of a physiological monitoring system according to the present invention, which may include a single sensor or a plurality of sensors.

### DETAILED DESCRIPTION

The present invention generally includes systems and methods for measuring and analyzing different types of physiological data, particularly electrophysiological data, including but not limited to EEG, EKG, EMG, EOG and biomechanical data relating to breathing and/or respiration, to provide further insight to the person's health and/or behavior. It is believed that the exemplary embodiments described herein provide numerous benefits over existing methods and devices. For example, the systems and methods described herein may provide one or more physiological sensors having improved portability and/or comfort such that the sensor(s) may be wearable or otherwise remain in contact with the person as the person moves around. By contact with a person, it is contemplated that the contact may be direct (e.g., direct contact with the person's skin or scalp) or may be indirect (e.g., contacting the person's clothing or is sufficiently close to the person such that electrophysiological signals or biomechanical data may be reliably detected). These sensors may be useable by a person during day-to-day activities (e.g., driving, exercising, walking, sleeping, etc.), for an extended period of time (e.g., throughout the day or night), or may be persistently worn or attached to the person. These improved sensors may be used to measure and/or analyze numerous different types of physiological data that were previously limited to measurements using in-patient techniques, methods, and/or devices that are cumbersome and uncomfortable to the person. Therefore, it is believed that the exemplary embodiments will enable a wide range of new systems and methods, particularly portable or wearable devices, for acquiring and analyzing physiological signals, particularly electrophysiological signals and biomechanical data relating to breathing and/or respiration. Furthermore, the exemplary embodiments of the present invention allow for improved physiological detection that can be made readily accessible to a consumer that is comfortable, easy-to-use, and inexpensive.

Data measured by the exemplary embodiments may be used to better understand different biometric factors within the person. Specifically, the data may be used to quantify and/or analyze physiological signals and/or changes within a person, for example, in a device suitable for self-monitoring, such as those used in connection with the quantified self movement. For example, such self-monitoring systems and devices may combine the improved sensors as described herein with a processing/computing arrangement for analyzing data acquired by the sensors, reference or normative databases of physiological data collected from a plurality of different individuals or from the same person sufficient to establish a replicable or statistically reliable data set, and a user interface for displaying information to the person and to provide useful information, for example, health related recommendations, to enable the person to make changes in his or her daily life to achieve desired health-related goals. Although the present invention is described herein for detecting and analyzing physiological signals from a person, it is contemplated that the present invention may be used in connection with a human, a mammal or any other animal having electrophysiological signals.

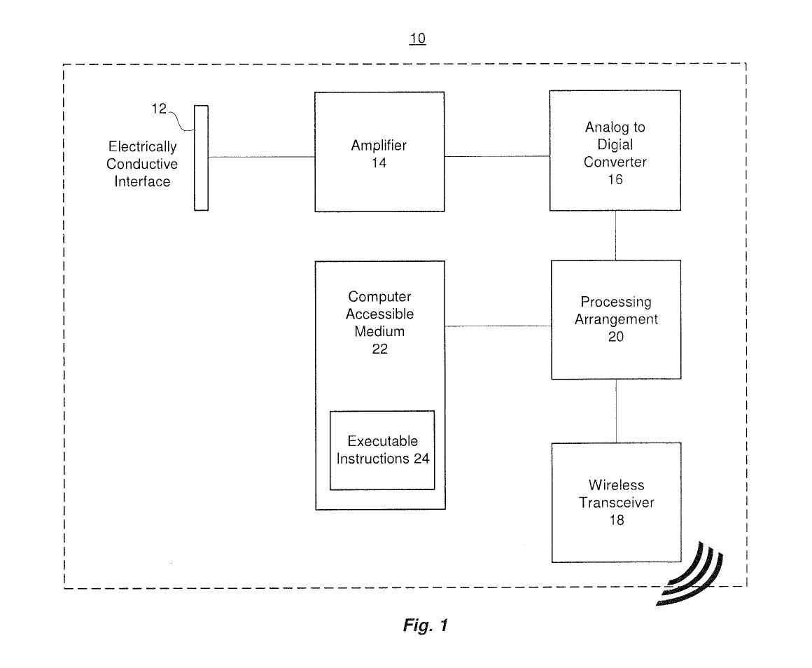

In one aspect of the present invention, a sensor 10 for detecting various different types of electrophysiological data or biomechanical data from a person is provided. FIG. 1 shows an exemplary embodiment of a sensor 10 according to the present invention. The sensor 10 may include an electrically-conducting interface 12 configured to be in contact with a person, for example, the skin of the person. In particular, the electrically-conducting interface 12 may be capable of detecting an electrophysiological signal without the use of an electrically-conducting gel. The electrically-conducting interface 12 may be suitable for obtaining (e.g., measuring from the person) any type of physiological data, in particular, electrophysiological data and/or biomechanical data. Examples of electrophysiological data include, but are not limited to, EEG, EKG (or ECG), EOG, and EMG. Electroencephalography (EEG) refers to the recording of the electrical activity of the brain over time. Electrocardiogram (EKG or ECG) refers to recording the electrical activity of the heart muscle over time. Electrooculogram (EOG) refers to the recording of eye muscle activity over time. Electromyogram (EMG) refers to the recording of the activity of skeletal muscles in the body over time.

The electrically-conducting interface 12 may also be suitable for measuring biomechanical data, in particular data relating to breathing and/or respiration of a person. Breathing (respiration) in humans is one of the most overt signs of life and an indicator of physiological status. As such, there is substantial value in measuring breathing in various situations and applications. Breathing and/or respiration of a person may be assessed and monitored using various types of electrophysiological sensors that may be activated by a mechanical pressure.

In certain embodiments, the electrically-conducting interface 12 may be activated by mechanical pressure. Therefore, the sensor 10 may also serve as a wireless mechanoelectrical sensor. The wireless mechanoelectrical sensor may be activated by physical motion by the person, for example, rolling over while asleep or breathing.

In some embodiments, the electrically-conducting interface 12 may be formed from a soft and pliable material, and thus, provide increased comfort when the electrically-conducting interface 12 is in contact with the person, particularly the skin of the person. Suitable materials for the electrically-conducting interface 12 may include, but is not limited to, electrically-conducting foam, fabric, thread, polymer, solid or liquid, metal, and fabric dyed with an electrically-conducting polymer.

The sensor 10, as shown in FIG. 1, may further include an amplifier 14, an analog-digital converter (ADC) 16, and a wireless transceiver 18. The electrically-conducting interface 12 may be in contact with the person and detect an electrophysiological signal and/or biomechanical data from the person. The electronically-conducting interface 12 may be connected to an amplifier 14, which receives the electrophysiological signal or biomechanical data detected by the electronically-conducting interface 12 and enhances the strength (e.g., increase the amplitude or decrease the impedance) of the electrophysiological signal and/or biomechanical data. The amplifier 14 may be connected to an analog-digital converter 16, which receives the amplified electrophysiological signal or amplified biomechanical data and converts the signal to digital data. The sensor 10 may further include an energy source, such as a battery (e.g., rechargeable or non-rechargeable) or capacitor that powers the sensors 10 and other electronic components. In one particular embodiment, the analog-digital converter 16 may be connected to a wireless transceiver 18, which receives the digitized data and wirelessly transmits the data to other sensors and/or a device or apparatus that records, stores, and/or processes data acquired by the sensor 10. The wireless transceiver 18 may also wirelessly transmit data concerning an operating status of the sensor 10. For example, the wireless transceiver 18 may wirelessly transmit data relating to a level of energy (e.g., level of battery charge) available from the energy source.

In an alternative embodiment, the analog-digital converter 16 may be connected to any suitable processing arrangement 20. The processing arrangement 20 may include a processor, e.g., one or more microprocessors, that direct the operation of the sensor 10. The processing arrangement 20 may direct the operation of the sensor 10 based on executable instructions 24 stored on a computer accessible medium 22 (e.g., memory or other data storage device). The digitized data may be stored by the processing arrangement 20 in the computer accessible medium 22 or a separate storage device within the sensor 10. In some embodiments, the computer accessible medium may be a low-power storage media, such as an SD card. The digitized data may be stored on the computer accessible medium 22 and may be wirelessly transmitted in parallel. In some embodiments, the digitized data may be stored for a predetermined period of time and periodically transmitted by a wireless transceiver 18, in a wireless manner, to other sensors and/or a device or apparatus that records, stores, and/or processes data acquired by the sensor 10. This local storage can be used to ensure data integrity and buffer signal transmission in the event that wireless signal transmission to the operating and signal processing component is interrupted. The wireless transceiver 18 may be capable of transmitting and/or receiving data. In one particular embodiment, the wireless transceiver 18 may wirelessly receive executable instructions, e.g., instructions directing operation of the sensor 10, for processing by the processing arrangement.

The wireless transceiver 18 may conduct wireless communications (e.g., transmit and/or receive data, executable instructions, and/or other information) via any suitable wireless link, (e.g., infrared, radio frequency, Bluetooth, IEEE 802.1x, etc.). In some embodiments, the wireless transceiver 18 may utilize communications links having a limited range, for example, near field communications (NFC).

In certain embodiments, the sensor 10 does not include physical wires extending therefrom and therefore, eliminating cumbersome wiring that are often difficult to use and uncomfortable for the person. In general, each sensor 10 may wirelessly communicate by any suitable means with other sensors or a device or apparatus that records, stores, and/or processes data acquired by the sensor 10 and/or other sensors. More particularly, each sensor 10 may be self-contained and not connected to any other sensor or device by a wired connection.

In a particular exemplary embodiment, the sensor 10 may be a self-contained sensor 10 about the size of a watch battery. Specifically, the sensor 10 may have an average diameter from about 1 cm to about 2 cm. It is therefore contemplated that each of the components of the sensor 10, e.g., the electrically conductive interface 12, amplifier 14, analog to digital converter 16, wireless transceiver 18, processing arrangement 20, and computer accessible medium 22, are each about or less than the size of the sensor 10 specified above.

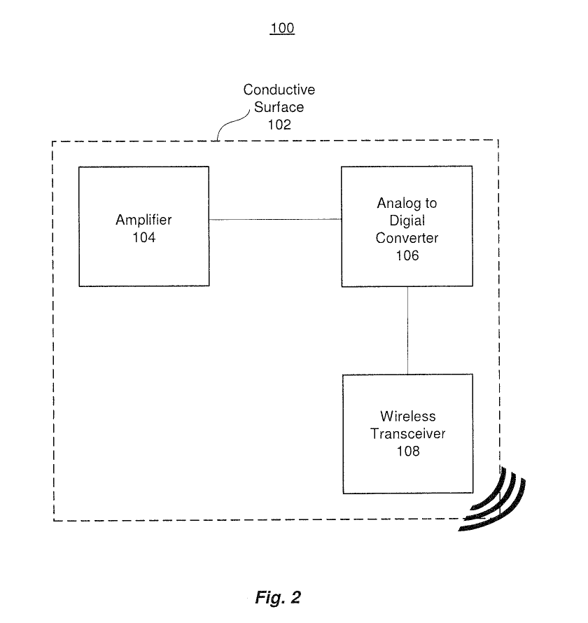

In an alternative embodiment, a sensor 100 for detecting various different types of electrophysiological data or biomechanical data from a person is shown in FIG. 2. The sensor 100 comprises a conductive surface 102 integrated into a soft and pliable material. In particular, the soft and pliable material may have a sheet form, such as, for example, a woven or non-woven fabric. More particularly, the soft and pliable material may provide a suitable electrical and mechanical substrate for housing a number of electrical circuits for obtaining electrophysiological or biomechanical data, for example, an amplifier 104 and an analog to digital converter 106. For example, the soft and pliable material may include conductive fibers or wires integrated therein such that the conductive surface 102 is electrically connected to the amplifier 104, and the amplifier 104 is electrically connected to the analog to digital converter 106. The conductive surface 102 may be in contact with the person, for example, the skin of the person, and detect an electrophysiological signal and/or biomechanical data from the person. The conductive surface 102 may be suitable for obtaining (e.g., measuring from the person) any type of physiological data, in particular, electrophysiological data and/or biomechanical data, such as those discussed above with respect to the electrically-conducting interface 12 of sensor 10. The electrophysiological signal or biomechanical data obtained by the conductive surface 102 may subsequently be enhanced in strength (e.g., increase in amplitude) by the amplifier 104. The amplified electrophysiological signal or amplified biomechanical data may be converted from an analog signal to digital data by the analog to digital converter 106.

In certain embodiments, the conductive surface 102 may be activated by mechanical pressure. Therefore, the sensor 100 may also serve as a wireless mechanoelectrical sensor. The wireless mechanoelectrical sensor may be activated by physical motion by the person, for example, rolling over while asleep or breathing.

In one exemplary embodiment, the sensor 100 may optionally further include a wireless transceiver 108 that is electrically connected via the conductive fibers or wires integrated within the soft and pliable material to the analog to digital converter 106. The wireless transceiver 108 may conduct wireless communications (e.g., transmit the obtained digital data) via any suitable wireless link, (e.g., radio frequency, Bluetooth, IEEE 802.1x, etc.). In some embodiments, the wireless transceiver 108 may utilize communications links having a limited range, for example, near field communications (NFC). The sensor 100 may further include an energy source, such as a battery (e.g., rechargeable or non-rechargeable) or capacitor that powers the sensors 100 and other electronic components.

In a particular exemplary embodiment, the conductive surface 102 may be a conductive node integrated into a fabric. The fabric may include a plurality of sensors 100 integrated therein, the plurality of sensors 100 forming a sensor array. Conductive fibers may be integrated into the fabric to form a fabric with conductive nodes distributed therethrough. Each of the conductive nodes may be electrically independent from each other, but may be individually electrically connected to a voltage processing unit, i.e., either the amplifier 104 or the analog to digital converter 106. Each conductive node may be paired with a designated amplifier 104 and a designated analog to digital converter 106, the designated amplifier 104 and the analog to digital converter 106 may be designated for each conductive node or shared among two or more conductive nodes. Alternatively, a plurality of conductive nodes may share a single voltage processing unit, i.e., either the amplifier 104 or the analog to digital converter 106. The conductive nodes may be connected to a voltage processing unit via any electrically conductive means, such as, for example, conductive fibers or wires integrated within the fabric. For example, the connection of each conductive node to a voltage processing unit is, in one embodiment, via conductive fiber tracts that are woven into the fabric or, in another embodiment, via galvanic wires that are integrated into the fabric or run between two or more layers of the fabric. The conductive nodes may have any suitable shape and may be distributed within the fabric in any suitable manner. In one embodiment, the conductive nodes are co-planar with a surface of the fabric. In another embodiment, the conductive nodes may have the shape of a bump or a nipple rising out of the surface of the fabric. More particularly, the conductive nodes may form a raised surface that is suitably configured for forming a relatively low (e.g., 5-100 kOhm) impedance pressure connection to the skin of the person that is being recorded.

In another exemplary embodiment, the conductive surface 102 may be a section of a quilted meshwork of a fabric containing conductive fibers. Each section of the quilted meshwork may form an independent conductive surface 102. More particularly, each section of the quilt may be electrically isolated from other sections of the quilt, but may be individually electrically connected to a voltage processing unit, i.e., either the amplifier 104 or the analog to digital converter 106. Each conductive surface 102 may be paired with a designated amplifier 104 and a designated analog to digital converter 106, the designated amplifier 104 and the analog to digital converter 106 may be designated for each conductive node or shared among two or more conductive nodes. Alternatively, a plurality of conductive surfaces 102 may share a single voltage processing unit, i.e., either the amplifier 104 or the analog to digital converter 106.

In one exemplary embodiment, the conductive nodes may be activated by mechanical pressure. For example, the conductive surface 102, such as, for example, the conductive nodes (e.g., bumps or nipples) integrated in the fabric, may function as a switch such that when the person's body presses sufficiently hard on the conductive surface 102 (and thereby forms a low impedance connection with the person) the mechanical pressure actives a conductive connection between the conductive surface 102 and a voltage processing unit. This particular exemplary embodiment may be formed into bed sheets configured to detect EEG, EKG, pulse and/or respiration from an individual.

The sensors 10 and 100 described above are merely illustrations of exemplary embodiments of the present invention, which is not to be limited in scope by the particular embodiments described herein. The embodiments shown above in FIGS. 1 and 2 are a subset of sensors that may be used to obtain mechanoelectrical and/or electrophysiological signals according to the present invention. Different embodiments utilize a wide variety of different materials for the electrically-conducting interface 12 or the conducting surface 102. Some embodiments comprise only one sensor while others comprise a plurality of sensors.

In an alternative exemplary embodiment, an electrophysiological sensor for acquiring physiological signals, and for wirelessly transmitting said physiological signals may be provided. The sensor may comprise an electrically conducting interface in contact with a person, an amplifier circuit, an analog-digital converter (ADC) circuit, and a wireless transceiver circuit. The physiological signals detected by the sensor may include at least one of EEG, ECG/EKG, EMG, EOG and breathing/respiration. The physiological signals may include at least one of voltage signals and/or capacitive signals. In some embodiments, the electrically conducting interface may comprise at least one of electrically-conducting gel, foam, fabric, polymer, solid, liquid. In other embodiments, the electrically conducting interface may comprise metal. The electrically conducting interface may comprise at least one of an electrically-conducting polymer foam or fabric. In particular, the electrically-conducting interface may comprise a fabric comprised of electrically conducting threads. The electrically conducting threads may comprise at least one of metal, electrically-conducting polymer, and fabric dyed with electrically-conducting polymer. The electrophysiological sensor may also comprise an energy source for powering the electronics, such as for example, a battery, a rechargeable battery, and/or capacitors. The wireless transceiver circuit may transmit status signals, wherein the status signals comprise at least one of the level of the energy source (e.g., battery charge) and/or loss of an expected signal. The wireless transceiver may also be configured to receive at least one of control, timing, reference and ground signals. The electrophysiological sensor may include a storage media or memory. The electrophysiological sensor may be activated by mechanical pressure and therefore, may include a mechanoelectrical sensor. The amplifier circuit may be configured to include a voltage source follower, whereby the sensed voltage is referred to the output of the operational amplifier.

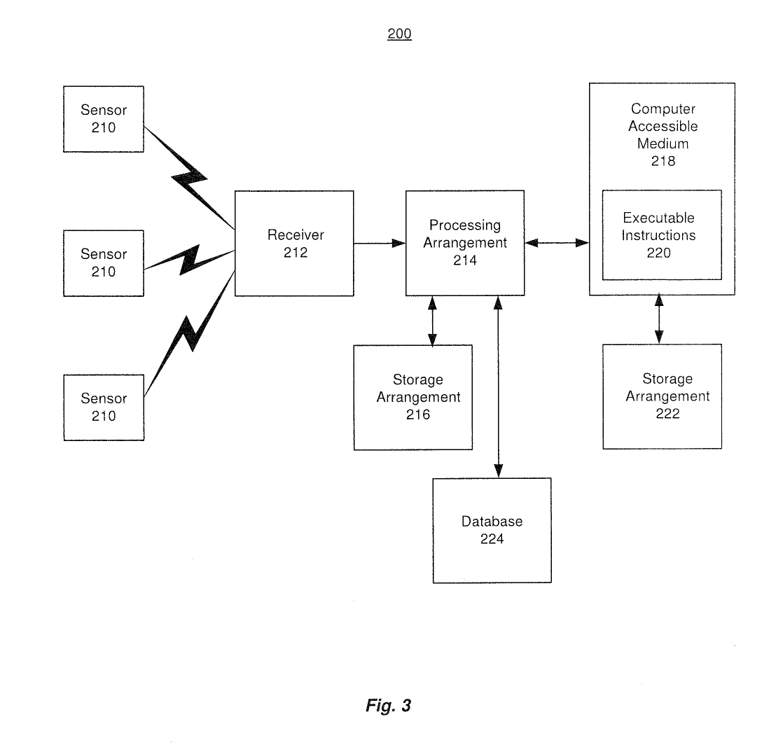

In another aspect of the present invention, a physiological monitoring system is provided. FIG. 3 shows an exemplary embodiment of a physiological monitoring system 200 according to the present invention. The physiological monitoring system 200 may include one or more sensors 210 for detecting various different types of electrophysiological data or biomechanical data from a person. The sensors 210 may be in any suitable form, including those exemplary embodiments described above with respect to FIGS. 1 and 2.

In other embodiments, the sensors 210 may include a plurality of sensor arrays. For example, the sensors 210 may include two or more physically and electrically separate sensor arrays configured to achieve complex mechanical geometries. In one exemplary embodiment, the sensors 210 may comprise a plurality of conductive surfaces 102, such as, for example, conductive nodes (e.g., bumps or nipples) integrated in a fabric, or a plurality of sections of a quilted meshwork of a fabric containing conductive fibers. The sensors may be organized in a planar sheet and formed into various fabrics, textiles, clothing and/or bedding items. The sensors may be integrated into a smart sheet, blanket, or pillow case that senses and measures breathing, pulse, cardiac cycle, brain waves, and/or other physiological parameters. In a particular exemplary embodiment, a first set of sensors 210 may be organized in a first sensor array integrated in a bed sheet or a blanket. The first set of sensors 210 may be used in combination with a second set of sensors 210 formed in a second sensor array integrated into a clothing item, such as a sock, a waistband of an undergarment or other item of clothing, e.g., a shirt, or wristband or headband, cap, hood etc. Each fabric, textile, clothing or sheet items may be a physically separate sensor array, each having its own local and unique topography. The combination of two or more of these items form a network of sensors 210 having varying topography in a system for detecting, identifying, analysis of a person's electrophysiological signals and biomechanical data, such as, for example, the person's bodily and brain functions.

In another embodiment, the fabric can be such that it provides conductive “channels” from one planar side of the fabric to the other side. In this way, it can be configured into clothing that allows electrical signals on points of the subject's skin to be communicated through the clothing. This would overcome the insulation that clothing would typically cause as in for example the clothing a person might wear to stay warm would insulate the torso from other electrical sensors such as those on the bed sheet. By making conductive channels in the fabric that is used to make the clothing, the bed sheet sensors can be in electrical communication with the subject's torso.

The sensors 210 may each form a communications link, preferably a wireless communications link (e.g., radio frequency, Bluetooth, IEEE 802.1x, etc.), with a receiver 212, for transmitting, preferably wirelessly, data corresponding to electrophysiological signals and/or biomechanical data to the receiver 212. Each sensor 210 may include an amplifier for increasing the strength, e.g., amplitude or lower impedance of the electrophysiological signals and/or biomechanical data. The sensor 210 may also include an analog to digital converter for digitizing the amplified data. In some embodiments, the wireless communications link between the sensors 210 and the receiver 212 may include communications links having a limited range, for example, near field communications (NFC). The receiver 212 may provide the received signals and/or data to a processing arrangement 214 for analysis. Those skilled in the art will understand that exemplary embodiments for analyzing the received signals and/or data may be implemented in any number of manners, including as a separate software module, as a combination of hardware and software, etc. For example, the exemplary embodiments for analyzing the received signals and/or data may be embodied in one or more programs stored in a non-transitory storage medium and containing lines of code that, when compiled, may be executed by at least one of the plurality of processor cores or a separate processor. In some embodiments, a system comprising a plurality of processor cores and a set of instructions executing on the plurality of processor cores may be provided. The set of instructions may be operable to perform the exemplary embodiments for analyzing the received signals and/or data discussed further below.

For example, the physiological monitoring system 200 may include a processing arrangement 214 for analyzing the received signals and/or data. The processing arrangement 210 may be, e.g., entirely or a part of, or include, but not limited to, a computer/processor that can include, e.g., one or more microprocessors, and use instructions stored on a computer-accessible medium (e.g., RAM, ROM, hard drive, or other storage device). As shown in FIG. 3, e.g., a computer-accessible medium 218 (e.g., as described herein, a storage device such as a hard disk, floppy disk, memory stick, CD-ROM, RAM, ROM, etc., or a collection thereof) may be provided (e.g., in communication with the processing arrangement 214). The computer-accessible medium 218 may be a non-transitory computer-accessible medium. The computer-accessible medium 218 can contain executable instructions 220 thereon. In addition, or alternatively, a storage arrangement 222 (e.g., storage memory) can be provided separately from the computer-accessible medium 218, which can provide the instructions to the processing arrangement 214 so as to configure the processing arrangement 214 to execute certain exemplary procedures, processes and methods, as described herein.

The processing arrangement 214 may be in communication with another storage arrangement 216 (e.g., storage memory) which can be used to store the received signals and/or data. The storage arrangement 216 may also be used to store outputs of analysis performed by the processing arrangement.

The physiological monitoring system 200 may further include an energy source, such as a battery (e.g., rechargeable or non-rechargeable) or capacitor that powers the sensors 210 and other electronic components. In some embodiments, the sensors 210 may transmit status signals, such as the level of the energy source (e.g., battery charge) or loss of an expected signal via its wireless transceiver to the receiver 212. In other embodiments, the sensors 210 may also convey a capacitive signal along with a voltage signal to a signal processor via its wireless transceiver to the receiver 212. As capacity is related to the geometric arrangement of two conductive surfaces and their separation, the capacitive signal provides an indicator of the mechanical force, presence or absence of pressure on the capacitive sensor.

A plurality of sensors of the present invention can be used to obtain and record a large amount of electrophysiological signals and biomechanical data from a person. However, yin some embodiments, the sensors may each operate independently and may not be in communication with each other. Therefore, use of a plurality of such independent sensors. e.g., wireless electrophysiological and/or mechanoelectrical sensors, face unique operation coordination challenges, such as, for example, grounding, timing, data quality management, and dynamically selecting subsets of sensors providing relevant data.

Any voltage sensor must refer the voltage that is being sensed to a standard voltage called the reference. In some embodiments, each electrode may be connected to a source follower amplifier (this also lowers impedance which reduces noise). At a later stage of processing, the source follower signals can be referred to one another or not for further amplification or other signal processing. In one exemplary embodiment, one or more of a plurality of sensors 210 in a physiological monitoring system 200 may be selected to be configured as a voltage source follower. In particular, the amplifier of the selected sensor(s) 210 may be configured as a voltage source follower, whereby the sensed voltage of the selected sensor is referred to by the amplifiers of the other remaining sensors 210. Because the sensors 210 only contact the person at a single galvanically isolated point, reconfiguring the sensors 210 as a source follower solves the problem that the sensors need two signal inputs in order to measure a voltage corresponding to electrophysiological signals of a person.

There is, however, a second problem to overcome in this embodiment—that is, a lack of a common ground. The energy sources, such as a battery, may be used to provide a grounding point for each sensor. However, the sensors 210 may each operate with an independent battery or other energy source that do not share a common ground. In this embodiment, each wireless sensor would operate independently but it is often valuable to a use a plurality of sensors, in which case each sensor's output voltage is floating—without a common grounding point. As discussed further below, there are multiple ways to overcome this lack of a common ground.

In one particular embodiment, the lack of a common ground may be resolved by shorting the grounds of the energy sources of each of the sensors so that all of the sensors share a common floating ground together. For example, the sensors may be integrated into a fabric, which may be in the form of a clothing item such as, for example, a cap, hood, sock or shirts, or a bedding item, such as a bed sheet or a blanket. While most of the fabric acts as an insulator, the fabric may be constructed with conductive threads so as to short the grounds of the energy sources of the individual sensors. In this way, each sensor floats relative to a point remote from the person being monitored, but all of the sensors float together.

In another embodiment, the lack of a common ground may be resolved by actively adjusting the ground for each of the plurality of sensors. For example, a transceiver circuit, such as the wireless transceiver of each sensor, may be used to receive data corresponding to a voltage adjustment up or down from a remote sensor controller. This voltage adjustment is used to actively adjust the ground of each of the sensors. This active ground configuration may be controlled by wirelessly sending the appropriate voltage adjustment to the sensor. The adjustment may be determined by any of a number of suitable means for obtaining a computed ground. The computed ground is an instantaneous or short duration estimate of the net voltage from the plurality of sensor outputs and/or the deviation of this voltage from a target voltage. The deviation or absolute value of this computed ground, once fed back to the individual sensors may be used to set the voltage adjustment of the active ground on each of the individual sensors.

In a further embodiment, the lack of a common ground may be resolved by configuring each sensor as part of a sensor pair such that the two sensor surfaces are organized so that one sensor provides the sensed voltage and the other sensor provides the reference and/or ground. The physical arrangement of the sensor pair, in one embodiment, is a center-surround arrangement where the center sensor senses the voltage of interest and the surrounding sensor senses the reference voltage. This solution works best when the surrounding sensor can be a significant distance from the center sensor as for instance if it were part of a fabric formed into a clothing item that resembled a skull cap, or hood worn on the head, where there is a signal sensor located in the center and at least one surrounding sensor located at the margins of the cap or hood.

Because the sensors may each operate independently and may not be in communication with each other, the plurality of sensors 210 in a physiological monitoring system 200 may not share a common time registration. This is a key problem in the use of more than one independently operated sensor in a coordinated system of sensors. As discussed further below, there are multiple ways to overcome this lack of a common time registration and establish a moment in time that is common to all the sensors.

In one embodiment, the plurality of sensors 210 may share a common signal processing clock. This can be established through the use of any number of timing devices, for example, use of phase-locked loop (PLL) circuits, access to which is shared by all the sensors.

In another embodiment, the plurality of sensors 210 may be put into a time register by each sensor 210 independently and wirelessly consulting a master clock, such as the atomic clock, or a local source for keeping time, such as a remote signal processing unit in the vicinity of the physiological monitoring system 200.

In a further embodiment, each of the plurality of sensors 210 may detect a common signal that is intrinsic to the person being recorded. For example, this common signal may be a heartbeat detected by an EKG. The heartbeat may be commonly detectable from many points of the person's body, including the head and limbs. Alternatively, respiratory, eye and body movements may be used as bioelectrical signal artifacts for adapting a common signal so that the plurality of sensors 210 may be adapted into a common time register.

The physiological monitoring system 200 described above merely illustrates exemplary embodiments of the present invention, which is not to be limited in scope by the particular embodiments described herein. In another exemplary embodiment, a physiological monitoring system for acquiring physiological signals, and for wirelessly transmitting said physiological signals may be provided. The physiological monitoring system may comprise at least one or a plurality of electrophysiological and/or mechanoelectrical sensors, at least one or a plurality of amplifier circuits, at least one or a plurality of analog-digital converter (ADC) circuits, at least one or a plurality of wireless transceiver circuits, at least one or a plurality of energy sources, a receiver circuit, a processor, and at least one or a plurality of memory circuits or storage media. The physiological signals may comprise at least one of EEG, ECG/EKG. EMG, EOG, and breathing/respiration. Each sensor may comprise a conductive node. Each conductive node may comprise one or a plurality of electrically-conducting interfaces in contact with a person. The conductive nodes may be co-linear with the plane of the fabric surface or may be in the form of nipples rising out of the plane of the fabric surface. The conductive nodes may also be electrically isolated from one another. Each conductive node may be electrically connected via conductive tracts to the amplifier, ADC, and/or wireless transceiver circuits. The at least one energy sources may power electronic components included within the physiological monitoring system. The energy sources may comprise at least one of a battery, a rechargeable battery, and capacitors. The receiver circuit may receive the physiological signals transmitted wirelessly from the at least one wireless transceiver circuits. The processor may perform processing of the physiological signals. The memory and storage media may store the physiological signals.

In some embodiments, the sensors may comprise energy sources configured so as to short the grounds of the energy sources (i.e., provide a common float). The sensor may comprise a wireless receiver configured so as to receive a computed ground. The computed ground may be at least one of an instantaneous or short duration estimate of a net voltage from the plurality of the sensor outputs, and the deviation of a net voltage from a target voltage. A deviation or absolute value of the computed ground may be used to set a voltage adjustment of an active ground for each of the sensors. The sensors may also be configured with at least two electrically-conducting interfaces. One of the electrically-conducting interfaces may provide at least one of the reference voltage and ground. In certain embodiments, the sensors may have a center-surround configuration. The sensors may be electrically connected via conductive tracts to a common clock. The sensors may also comprise a wireless receiver configured so as to receive timing signals from a master clock. The sensors may utilize detection of a common timing signal that may be intrinsic to the person that is being recorded. In particular, each sensor may comprise a signal processor, where the signal processor extracts the common timing signal by processing at least one of EEG, ECG/EKG, EMG, EOG, and breathing/respiration.

In another aspect of the present invention, systems and methods for analyzing physiological data may be provided. For example, the processing arrangement 214 may be used to analyze physiological signals and/or data obtained by the sensors 210. The processing arrangement 214 may analyze the physiological data to perform a number of functions including: determining data quality, removing noise, rejecting bad quality data, detecting and removing artifacts, and separating the physiological signals into different components (e.g., EEG, EKG, EMG, EOG, respiration). The processing arrangement 214 may also perform processing of physiological data or signals, including, for example, at least one of: data quality management; determining data quality; detecting artifacts; attenuating/eliminating noise; identifying features of interest; distinguishing and identifying those sensors with useful information from those other sensors that carry no useful information; pattern recognition; pattern classification; separating at least two of EEG, ECG/EKG, EOG, EMG, breathing/respriation signals, noise, and artifacts; recognizing or identifying a physiological state; comparing the physiological signals acquired from one individual with one or a plurality of such physiological signals acquired from the same individual at different times; comparing the physiological signals acquired from one individual with one or a plurality of such physiological signals acquired from different individuals at the same or different times; determining a person's body shape; and identifying the locations on a person's body from which said physiological signals emanate. Conventional devices for recording physiological signals were designed to avoid or minimize the need for such analysis functions. However, in the physiological monitoring system 200 shown in FIG. 3 such systems and methods for analyzing physiological data may be particularly beneficial, for example, the data may be used to analyze the health and/or behavior of a person, and/or may recommend changes that are believed to improve the health of the person.

The processing arrangement 214 may be used to conduct a number of different methods for signal processing and data analysis, including but not limited to data quality management, determine data quality, detect artifacts, attenuate/eliminate noise, identify features of interest, and to separate EEG, EKG, EOG, EMG, and breathing/respiration signals. Such signal processing methods include temporal filtering, nonlinear filtering (e.g., median filtering, anisotropic diffusion), regularization, spectral analysis, computing a Fourier transform, computing a power spectrum, computing a Hilbert transform, computing a discrete cosine transform, computing a subband transform, computing a wavelet transform, computing band-limited power, comparing band-limited power in a plurality of frequency bands, computing a phase locking factor, computing of cross-frequency coupling, feature detection, arithmetic operations (e.g., addition, subtraction, multiplication, division) applied to a plurality of signals, vector product between two signals, projection, principal components analysis (PCA), singular value decomposition (SVD), factor analysis, independent components analysis (ICA). These signal processing methods are applied to analyze the semi-instantaneous as well as prolonged frequency content of the signals at a single, as well as arrangements of a plurality of sensors. Embodiments of the invention are not limited to the signal processing operations listed above, which are given as a subset of the signal processing operations that can be applied to process the mechanoelectrical and electrophysiological signals obtained by sensors 210.

The processing arrangement 214 may also be used to extract one or more temporal epochs from physiological signals and perform at least one of signal processing operations and comparison operations on each of said epochs. Such signal processing methods may include, for example, temporal filtering, nonlinear filtering (e.g., median filtering, anisotropic diffusion), regularization, spectral analysis, computing a Fourier transform, computing a power spectrum, computing a Hilbert transform, computing a discrete cosine transform, computing a subband transform, computing a wavelet transform, computing band-limited power, comparing band-limited power in a plurality of frequency bands, computing a phase locking factor, computing a cross-frequency coupling, feature detection, arithmetic operations (e.g., addition, subtraction, multiplication, division) applied to a plurality of signals, vector product between two signals, projection, principal components analysis (PCA), singular value decomposition (SVD), factor analysis, and independent components analysis (ICA); and wherein said comparison operations comprising at least one of difference (i.e., subtraction), ratio (i.e., division), correlation, canonical correlation, sum of squared difference, least-squares, partial least squares, nearest neighbor, Mahalonobis distance, regression, multiple linear regression, logistic regression, polynomial regression, general linear model, support vector machine regression, principal components analysis (PCA), singular value decomposition (SVD), factor analysis, independent components analysis (ICA), multidimensional scaling, and/or dimensionality reduction.

In one exemplary embodiment, signal processing methods may be used to distinguish and identify those sensors with useful information from those other sensors that carry no useful information. For example, the output of a sensor that has not changed beyond a threshold value can be deemed non-detecting and as such is indicative that the person is not in contact with that sensor. In another example, sensors that provide signals above certain pre-selected thresholds, such as a threshold where the analog to digital converter saturates, may be considered as uninformative and of sufficiently poor quality such that they are ignored in any analysis or signal processing.

The processing arrangement 214 may also perform pattern recognition and pattern classification operations to recognize or identify various physiological states. For example, physiological states may include the different stages of sleep. Pattern recognition operations may include correlation, canonical correlation, sum of squared difference, least-squares, partial least squares, nearest neighbor, Mahalonobis distance, regression, multiple linear regression, logistic regression, polynomial regression, general linear model, principal components analysis (PCA), singular value decomposition (SVD), factor analysis, principal components regression, independent components analysis (ICA), multidimensional scaling, dimensionality reduction, maximum likelihood classifier, maximum a posteriori classifier, Bayesian classifier, Bayesian decision rule, radial basis functions, linear discriminant analysis, regularized discriminant analysis, general linear discriminant analysis, flexible discriminant analysis, penalized discriminant analysis, mixture discriminant analysis, Fischer linear discriminant, regularization, density estimation, naive Bayes classifier, mixture model, Gaussian mixtures, minimum description length, cross-validation, bootstrap methods, EM algorithm, Markov chain Monte Carlo (MCMC) methods, regression trees, classification trees, boosting, AdaBoost, gradient boosting, neural network classifier, projection pursuit, projection pursuit regression, support vector machine, support vector classifier, K-means clustering, vector quantization, k-nearest-neighbor classifier, adaptive nearest-neighbor classifier, cluster analysis, clustering algorithms, k-medoids, hierarchical clustering, sparse principal components, non-negative matrix factorization, nonlinear dimension reduction, undirected graph models, machine learning, statistical learning, supervised learning, and unsupervised learning. Embodiments of the invention are not limited to the pattern recognition operations listed above, which are given as a subset of the pattern recognition operations that can be applied to process the mechanoelectrical and electrophysiological signals obtained by sensors 210.

In one exemplary embodiment, signal processing methods may include analysis of the spatial arrangement of the plurality of sensors 210. In one exemplary embodiment, the processing arrangement 214 may analyze the data obtained from the sensors 210 and their spatial arrangement to determine a body shape of a person, such as a sleeping individual that may be reclined on a fabric integrated with a plurality of sensors 210 or wearing clothes formed from a fabric integrated with the plurality of sensors 210. The processing arrangement 214 may process the capacitive and/or voltage signals generated by each of the sensors 210.

For example, the processing arrangement 214 may analyze the electrophysiological signals and/or biomechanical data obtained from the plurality of sensors 210 in conjunction with their spatial arrangement to determine the body shape, or locations on a person's body from which the physiological signals emanate. This analysis may be similar to signal processing used to perform image and shape recognition from visual digital electronic images. Such processes may utilize prior knowledge and expectations of what constitutes a body shape and how that best fits the pattern of provided by the data retrieved by the plurality of sensors 210. In some embodiments, the analysis for a body shape may be directly analogous to a process for identification of a visual image in the array of pixel elements in detecting visual images in a digital camera's image sensor. The processing arrangement 214 in the present invention analyzes electrophysiological and mechanoelectric data instead of a visual intensity pattern.

In an exemplary embodiment, this spatial information may be used to intelligently and dynamically select appropriate subsets of sensors to analyze for localizing and identifying physiological signals that emanate from a desired anatomical region, e.g., the head region, the chest region, the limbs, and so on. In one particular embodiment, the processing arrangement 214 may rapidly and serially sample the signals obtained by multiple sensors 210 at different spatial locations using an electronic switching scheme. After one or more such scan cycles, the processing arrangement 214 may determine the appropriate subset of sensors 210 to sample at higher temporal resolution for physiological signal monitoring and analysis. When the signals themselves and/or their quality changes sufficiently, the scan cycle may be repeated to establish a new best subset of sensors 210 for close monitoring. In this way, the processing arrangement 214 intelligently selects the most informative subset of the sensors 210 to analyze and to optimize useful and/or actionable information obtained and processed by the physiological monitoring system 200.

In a further embodiment, the processing arrangement 214 may analyze the electrophysiological signals and/or biomechanical data obtained from the plurality of sensors 210 in conjunction with their spatial arrangement along with concurrent visual images of the person. In this particular embodiment, image analysis and visual identification of the body configuration and parts may be used to constrain and guide algorithmic interpretations of the data obtained by the sensors 210 to identify the body configuration and further analyze the electrophysiological signals and biomechanical data obtained by the sensors 210.

In some embodiments, the processing arrangement 214 may also separate EEG, EKG, EOG, EMG, and/or breathing/respiration signals. For example, the processing arrangement 214 may utilize various methods for signal processing, pattern recognition, and/or body shape operations, as described above, applying these methods individually or in conjunction to each electrophysiological signal or each type of biomechanical data. In one exemplary embodiment, the electrophysiological signals (e.g., EEG) obtained by sensors 210 deemed to be in contact with the head may be selected for localization, identification and analysis. In another embodiment, the electrophysiological signals (e.g., EKG) obtained by sensors 210 deemed to be in contact or near the left, right, and lower margins of the pericardial chest cavity (i.e., sites most closely approximating Einthoven's triangle) may be selected for localization, identification and analysis. In another embodiment, the signals obtained by sensors 210 deemed to be proximal to the chest and/or diaphragm is selected for localization, identification and analysis of breathing. In a further embodiment, the signals (e.g., EMG and/or associated movement of the relevant body parts) obtained by sensors 210 deemed to be proximal to the chest, limbs or other body parts may be selected for localization, identification and analysis. In another embodiment, the signals (e.g., of eye movement and/or jaw clenching and movement) obtained by sensors 210 deemed to be proximal to the periorbital regions of the head, and/or the masticating muscles of the head may be selected for localization, identification and analysis. In another embodiment, the signals obtained by sensors 210 deemed to be proximal to unidentified and/or particular body parts may be selected for localization, identification and analysis of movement because during movement the local and/or global pattern of signals at these sensors may change with identifiable signal amplitudes, frequencies and/or mechanoelectrical statistics.

These as well as other signals may be used in embodiments of the present invention to improve signal quality and analysis of target physiological signals. For example, identification of the EKG from the sensors at the chest region may be used to identify EKG signals that contaminate EEG signals obtained from sensors proximal to the head. The contaminating EKG signals may then be identified and ignored, filtered out or otherwise eliminated in analysis of EEG obtained from sensors proximal to the head. Furthermore, the presence of EMG signals localized to appropriate sensors may be used to identify projections of these signals that contaminate other signals of interest, such as the EKG or breathing.

Embodiments of the invention are not limited to the examples described above, which are given as a subset of the signals that can be extracted from the sensor array of mechanoelectrical and electrophysiological signals.

Some embodiments of the present invention comprise a method for comparing physiological signals acquired from one individual with a plurality of such physiological signals acquired from the same individual at different times, or for comparing the physiological signals acquired from one individual with one or a plurality of such electrophysiological signals acquired from different individuals at the same or different times. The method may be performed by the processing arrangement 214. Comparison operations include difference (i.e., subtraction), ratio (i.e., division), correlation, canonical correlation, sum of squared difference, least-squares, partial least squares, nearest neighbor, Mahalonobis distance, regression, multiple linear regression, logistic regression, polynomial regression, general linear model, support vector machine regression, principal components analysis (PCA), singular value decomposition (SVD), factor analysis, independent components analysis (ICA), multidimensional scaling, dimensionality reduction, pattern recognition, and pattern classification (pattern recognition and pattern classification operations include those listed above).

Further, the processing arrangement 214 may optionally be in communication with a database 224. The database 224 may contain reference physiological data collected from a plurality of different individuals or from the same person sufficient to establish a replicable or statistically reliable data set. The reference physiological data may be collected at different times. A portion of or the entirety of the database 224 may be used by the processing arrangement 214 in analyzing the received signals and/or data.

In some embodiments, physiological signals acquired by the plurality of sensors 210, in raw and/or processed form, may be collected and stored in one or a plurality of databases 224. Physiological signals and/or corresponding data may be stored in the database 224 in association with metadata, which may include user identity and metrics, date and time, user notes, user history, demographic, and/or social information. In some embodiments, physiological signals and/or corresponding data may be stored and accumulated in the database 224. In this particular embodiment, the physiological signals and/or corresponding data collected by the plurality of sensors 210 may be added to the database 224 as part of the reference physiological data.

In some embodiments, the reference physiological data may be used to provide likelihood estimates of normal and/or abnormal body or physiological function as assessed by comparing the physiological signals and data measure from one individual with those provided as reference physiological data stored in the database 224. In some embodiments, the comparison is restricted to individuals who share particular common metadata characteristics (e.g., age). In some embodiments, the comparison may be restricted based on the user's particular interests.

In a particular exemplary embodiment, the physiological signals and data measured from a person and the reference physiological data stored in the database 224 may be used and communicated in a social network. More particularly, the present invention may further include methods to promote social networking for sharing and comparison of physiological signals and data.

In another exemplar embodiment, the processing arrangement 214 may be used to provide useful information, for example, health related recommendations, to enable the person to make changes in his or her daily life to achieve desired health-related goals.

The physiological monitoring system 200 may further comprise a user interface for displaying information, including measured data and/or analysis, to the person. The user interface may display useful information, for example, health related recommendations, to enable the person to make changes in his or her daily live to achieve desired health-related goals. In one embodiment, the extracted and identified physiological signals may be visualized and communicated to a person via the user interface. For example, the user interface may be generated by a control module remote from the sensors 210, for example, the processing arrangement 214. In another embodiment, the user interface may be provided on a telecommunications device including but not limited to a computer monitor, mobile device such as a tablet, smart phone, iPad, iPhone and other such devices. The extracted and identified physiological signals and data may be provided via the user interface in real-time, in compressed and/or expanded time, according to the user's interest and need.

Suitable user interfaces may include, for example, at least one of a mobile phone (or other such telecommunications device) app, a web app, status signals and alerts to indicate periods of normal and/or abnormal sensor function, status signals and alerts to indicate periods of normal and/or abnormal physiological function, status signals and alerts to indicate physiological states, a means to select sensor subsets that are currently relevant, a means to configure sensor networks, and a means to control the operating parameters of said system.

In an embodiment of the invention, data about body and/or brain function such as sleep, breathing, EEG signals and the like, may be presented via the user interface for entertainment and/or information. For example, breathing may be communicated by an oscillating color sequence of sound tones such as a bass drum rhythm, while EEG may be communicated by a combination of higher frequency line traces and/or color displays and other musical notes so as to create a multisensory presentation of the person's physiological state.

A further embodiment may include status signals and alerts to indicate periods of normal and/or abnormal function. For example, the processing arrangement 215 may be configured by the user to signal sensor dysfunction, a person's movement, states of arousal, states of sleep and sleep quality, apnea and tachypnea, states of brain function such as sleep and awake, and states of cardiac function that are of interest to the user.

In another embodiment, the user interface may provide means to select sensor subsets that are currently relevant and configure sensor networks, as well as control the operating parameters of the invention.

The physiological monitoring system 200 as described above may be incorporated in a number of suitable devices. For example, the physiological monitoring system 200 may be incorporated in a number of consumer devices, such as but not limited to a baby sleep monitor, adult sleep monitor, fitness monitor, breathing and respiration rate monitor, pulse rate monitor, cardiac fitness monitor, alertness/arousal monitor (for example, while driving), physical performance/training monitors for athletes, mindfulness and other mental performance/training monitors. As another example, the physiological monitoring system 200 may be incorporated in a number of medical devices, such as devices for detection of being at risk of and/or diagnosis of and/or monitoring of a wide variety of medical conditions including sleep disorders, epilepsy, cardiac disease, respiratory disease, movement disorders, developmental disabilities (e.g., including autism), mental illnesses, neurological diseases, sudden infant death syndrome (SIDS), concussion diagnosis, traumatic brain injury diagnosis. The physiological monitoring system 200 may be incorporated in a number of commercial devices, such as, for example, alertness/arousal monitors for professional (e.g., truck) drivers, pilots, train operators, etc.; alertness/arousal monitors for security personnel (e.g., TSA, building security, casino security), sports concussion alerting and monitoring. The physiological monitoring system 200 may be incorporated in a number of military devices including but not limited to alertness/arousal monitors, physical and mental performance monitors, concussion alerting and monitoring.

The physiological monitoring system 200 may be incorporated into any suitable device for monitoring electrophysiological signals and/or biomechanical data, for example, a sleep monitor, a smart sheet, a baby monitor, a fitness monitor, a breathing and respiration monitor, a pulse rate monitor, a cardiac fitness monitor, and alertness/arousal monitor, a physical performance/training monitor for athletes, a mindfulness or other mental performance/training monitor, a monitor for sleep disorders, a monitor for epilepsy, a monitor cardiac disease, a monitor for respiratory disease, a monitor for movement disorders, a monitor for developmental disabilities including autism, a monitor for mental illnesses, a monitor for neurological diseases, a monitor for sudden infant death syndrome, a monitor for concussion, and a monitor for traumatic brain injury.

In one particular exemplary embodiment, the physiological monitoring system 200 may be used to detect an awake or a sleep state of a person. More particularly, the physiological monitoring system 200 may used to detect whether a baby is in an awake or a sleep state. In this particular embodiment, the physiological monitoring system 200 may be incorporated in a baby sleep monitor, in which the plurality of sensors 210 are integrated within a fabric. Specifically, the fabric may comprise a blend of cotton thread and an electrically-conducted thread and may be incorporated in a crib sheet for use with the baby. In a particular embodiment, the physiological monitoring system 200 may be an EEG baby sleep monitor—the sensors 210 may specifically measure and record EEG data of the baby to determine if the baby is awake or asleep.

Sleep is a major problem for parents of young children. This is demonstrated by the large number of best-selling books on “sleep training” geared toward new parents, and by the numerous courses offered to new parents focusing on helping them get a better handle on their infant's sleep. But despite the attention given to educating parents about how to teach their children to sleep, getting a good night sleep remains one of the most anxiety-provoking aspects of parenting. There are a large number of baby monitors currently on the market, but none provide direct information about sleep. The majority of devices provide an audio monitor so that parents can hear whether their baby is crying. But sound is a poor proxy for sleep. The absence of crying does not mean that an infant is necessarily asleep. Nor does audible crying necessarily indicate that a baby is awake and needs attention. A smaller number of baby monitors provide a video feed so that parents can watch their baby sleep. But video is also a poor solution because movement is not a proxy for sleep. A baby can move while asleep and a baby can be motionless while awake. Moreover, video monitoring requires that parents constantly observe the streaming video feed. A few baby monitors provide information about pulse rate or motion activity, but neither of these measures is a suitable proxy for sleep. Consequently, parents lack a reliable way to monitor the timing and quality of their babies' sleep. Sleep is an active brain state that is associated with distinct patterns of brain activity. A proper way to monitor sleep is by measuring the electrical activity of the brain. EEG is an accurate measure of sleep brain activity. However, to date an EEG baby sleep monitoring device that is a) easy to use; b) safe; c) delivers high fidelity signals; and d) interprets the data in a clear way to provide actionable information for parents, has been unavailable.

The physiological monitoring system 200 of the present invention solves this problem with an EEG baby sleep monitor for use at home by parents. In one embodiment, small, soft wireless electrodes or sensors 210 may be embedded in an attractive and easy-to-use sleep cap. In another embodiment, the sensors 210 may be integrated in a fabric that is formed into an article of clothing, like an onesie with a hood that touches the baby's head. In yet another embodiment, the physiological monitoring system 200 may be incorporated in a crib sheet that touches the baby's head. The physiological signals and/or data from the plurality of sensors 210 may be wireless transmitted to a receiver 212 and a processing arrangement 214, both of which may be part of a parent's computer or hand-held devices (e.g., iPhones, iPads, etc.). In another embodiment, the physiological signals and/or data may be uploaded to a database 224 containing reference physiological data corresponding to a plurality of such physiological signals acquired from a number of sleeping infants. In return, parents may be provided with useful information about their baby's sleep by comparing EEG recordings from their baby with reference or normative data stored in the database 224. The reference or normative data may reflect physiological data from a plurality of other individuals, particularly babies. In one embodiment, statistical analysis and machine learning methods automatically identify the various stages of sleep, and make predictions about when a child is likely to fall asleep, and for how long they will remain asleep, and assess the quality of their sleep. In another embodiment, the parents or other users may be engaged in a social network and share tips and scientific advice about sleep. In a further embodiment, the database of physiological signals acquired from sleeping infants is a normative database of human brain development. In one embodiment, the physiological signals acquired from each individual are compared with the normative database to detect individuals that are at risk of developmental disabilities including autism.

Another embodiment of the physiological monitoring system 200 is a physical performance and athletic training monitor in which EMG sensors are embedded in articles of clothing. In particular, the articles of clothing may be made from athletic performance fabrics.

In a particular exemplary embodiment, a sleep monitor may be provided. The sleep monitor may comprise at least one or a plurality of electrophysiological and/or mechanoelectrical sensors; at least one or a plurality of amplifier circuits; at least one or a plurality of analog-digital converter (ADC) circuits; at least one or a plurality of wireless transceiver circuits; at least one or a plurality of energy sources; a receiver circuit; a processor; and at least one or a plurality of memory circuits or storage media. The physiological signals may comprise at least one of EEG, ECG/EKG, EMG, EOG, and breathing/respiration. Each of the sensors may comprise a conductive node. Each conductive node may comprise one or a plurality of electrically-conducting interfaces in contact with a person. The conductive nodes may be co-linear with the plane of the fabric surface, or may be in the form of nipples rising out of the plane of the fabric surface. The conductive nodes may be electrically isolated from one another. Each of the conductive nodes may be electrically connected via conductive tracts to the amplifier, ADC, and/or wireless transceiver circuits. The energy sources may power electronic components of the sleep monitor. Each of energy source may comprise at least one of a battery, a rechargeable battery, and capacitors. The receiver may receive physiological signals transmitted wirelessly from the wireless transceiver circuits. The processor may perform processing of the physiological signals. The processing may comprise at least one of: data quality management; determining data quality; detecting artifacts; attenuating/eliminating noise; identifying features of interest; distinguishing and identifying those sensors with useful information from those other sensors that carry no useful information; pattern recognition; pattern classification; separating at least two of EEG, ECG/EKG, EOG, EMG, breathing/respiration signals, noise, and artifacts; recognizing or identifying a physiological state; comparing the physiological signals acquired from one individual with one or a plurality of such physiological signals acquired from the same individual at different times; comparing the physiological signals acquired from one individual with one or a plurality of such physiological signals acquired from different individuals at the same or different times; determining a person's body shape; and identifying the locations on a person's body from which said physiological signals emanate. In some embodiments, the electrically-conducting interface of the sleep monitor may comprise at least one of a sheet comprising electrically-conducting fabric, a pillow case comprising electrically-conducting fabric, and a blanket comprising electrically-conducting fabric. The processing may comprise determining a stage of sleep. The memory or storage media may store a sleep log comprising information about the total amount of sleep and the amount of time for each stage of sleep. The sleep monitor may also include a database for storing a plurality of physiological signals acquired from the same or different individuals at different times. The sleep monitor may further comprise a user interface. The user interface may include, for example, at least one of a mobile phone (or other such telecommunications device) app, a web app, status signals and alerts to indicate periods of normal and/or abnormal sensor function, status signals and alerts to indicate periods of normal and/or abnormal physiological function, status signals and alerts to indicate physiological states, a means to select sensor subsets that are currently relevant, a means to configure sensor networks, and a means to control the operating parameters of said system. The sleep monitor may also be configured to execute a process that provides recommendations to an individual to improve his/her sleep.

In another particular exemplary embodiment, a smart sheet may be provided. The smart sheet may comprise at least one or a plurality of electrophysiological and/or mechanoelectrical sensors; at least one or a plurality of amplifier circuits; at least one or a plurality of analog-digital converter (ADC) circuits; at least one or a plurality of wireless transceiver circuits; at least one or a plurality of energy sources; a receiver circuit; a processor; and at least one or a plurality of memory circuits or storage media. The physiological signals may comprise at least one of EEG, ECG/EKG, EMG, EOG, and breathing/respiration. Each of the sensors may comprise a conductive node. Each conductive node may comprise one or a plurality of electrically-conducting interfaces in contact with a person. The conductive nodes may be co-linear with the plane of the fabric surface, or may be in the form of nipples rising out of the plane of the fabric surface. The conductive nodes may be electrically isolated from one another. Each of the conductive nodes may be electrically connected via conductive tracts to the amplifier, ADC, and/or wireless transceiver circuits. The energy sources may power electronic components of the smart sheet. Each of energy source may comprise at least one of a battery, a rechargeable battery, and capacitors. The receiver may receive physiological signals transmitted wirelessly from the wireless transceiver circuits. The processor may perform processing of the physiological signals. The processing may comprise at least one of: data quality management; determining data quality; detecting artifacts; attenuating/eliminating noise; identifying features of interest; distinguishing and identifying those sensors with useful information from those other sensors that carry no useful information; pattern recognition; pattern classification; separating at least two of EEG, ECG/EKG, EOG, EMG, breathing/respiration signals, noise, and artifacts; recognizing or identifying a physiological state; comparing the physiological signals acquired from one individual with one or a plurality of such physiological signals acquired from the same individual at different times; comparing the physiological signals acquired from one individual with one or a plurality of such physiological signals acquired from different individuals at the same or different times; determining a person's body shape; and identifying the locations on a person's body from which said physiological signals emanate. The memory or storage media may store a log of physiological states, which may include, for example, at least one of a person's movement, states of arousal, states of sleep and sleep quality, states of respiration including apnea and tachypnea, states of brain function such as sleep and awake, and states of cardiac function. The smart sheet may also comprise a database for storing a plurality of physiological signals acquired from the same or different individuals at different times. The smart sheet may further comprise a user interface, such as, for example, at least one of a mobile phone (or other such telecommunications device) app, a web app, status signals and alerts to indicate periods of normal and/or abnormal sensor function, status signals and alerts to indicate periods of normal and/or abnormal physiological function, status signals and alerts to indicate physiological states, a means to select sensor subsets that are currently relevant, a means to configure sensor networks, and a means to control the operating parameters of said system.

In a further particular exemplary embodiment, a baby monitor may be provided. The baby monitor may comprise at least one or a plurality of electrophysiological and/or mechanoelectrical sensors; at least one or a plurality of amplifier circuits; at least one or a plurality of analog-digital converter (ADC) circuits; at least one or a plurality of wireless transceiver circuits; at least one or a plurality of energy sources; a receiver circuit; a processor; and at least one or a plurality of memory circuits or storage media. The physiological signals may comprise at least one of EEG, ECG/EKG, EMG, EOG, and breathing/respiration. Each of the sensors may comprise a conductive node. Each conductive node may comprise one or a plurality of electrically-conducting interfaces in contact with a person. The conductive nodes may be co-linear with the plane of the fabric surface, or may be in the form of nipples rising out of the plane of the fabric surface. The conductive nodes may be electrically isolated from one another. Each of the conductive nodes may be electrically connected via conductive tracts to the amplifier, ADC, and/or wireless transceiver circuits. The energy sources may power electronic components of the baby monitor. Each energy source may comprise at least one of a battery, a rechargeable battery, and capacitors. The receiver may receive physiological signals transmitted wirelessly from the wireless transceiver circuits. The processor may perform processing of the physiological signals. The processing may comprise at least one of: data quality management; determining data quality; detecting artifacts; attenuating/eliminating noise; identifying features of interest; distinguishing and identifying those sensors with useful information from those other sensors that carry no useful information; pattern recognition; pattern classification; separating at least two of EEG, ECG/EKG, EOG, EMG, breathing/respiration signals, noise, and artifacts; recognizing or identifying a physiological state; comparing the physiological signals acquired from one individual with one or a plurality of such physiological signals acquired from the same individual at different times; comparing the physiological signals acquired from one individual with one or a plurality of such physiological signals acquired from different individuals at the same or different times; determining a person's body shape; and identifying the locations on a person's body from which said physiological signals emanate. The memory or storage media may store a sleep log comprising information about the total amount of sleep and the amount of time for each stage of sleep. Alternatively, or in addition, the memory or storage media may store a log of physiological states. The physiological state may include, for example, at least one of a baby's movement, states of arousal, states of sleep and sleep quality, states of respiration including apnea and tachypnea, states of brain function such as sleep and awake, and states of cardiac function.

In some embodiments, the baby monitor may process physiological data to determine a stage of sleep. The electrically-conducting interfaces of the baby monitor may comprise at least one of a crib sheet comprising electrically-conducting fabric, and an article of clothing comprising electrically-conducting fabric. The baby monitor may also be configured to execute a process that provides recommendations to parents to improve their baby's sleep. The baby monitor may also include a database for storing a plurality of physiological signals acquired from the same or different individuals at different times. The baby monitor may further include a user interface, such as, for example, at least one of a mobile phone (or other such telecommunications device) app, a web app, status signals and alerts to indicate periods of normal and/or abnormal sensor function, status signals and alerts to indicate periods of normal and/or abnormal physiological function, status signals and alerts to indicate physiological states, a means to select sensor subsets that are currently relevant, a means to configure sensor networks. In certain embodiments, the baby monitor may comprise at least one of audio and video monitoring.

The invention described and claimed herein is not to be limited in scope by the specific embodiments herein disclosed since these embodiments are intended as illustrations of several aspects of this invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. All publications cited herein are incorporated by reference in their entirety.

1. A sensor for detecting physiological signals in a person comprising:
an electrically-conducting interface configured to be placed in contact with the person to detect an electrophysiological signal or biomechanical data from the person; and
a wireless transceiver configured to transmit data corresponding to the electrophysiological signal to a receiver external to the sensor, wherein the sensor does not include a wire extending therefrom connecting the sensor to any other electric device.

2. The sensor of claim 1, wherein the electrophysiological signal is selected from the group consisting of electroencephalography (EEG), electrocardiogram (EKG or ECG), electromyogram (EMG), and electrooculogram (EOG).

3. The sensor of claim 1, wherein the biomechanical data corresponds to breathing or respiration of the person.

4. The sensor of claim 1, wherein the electrically-conducting interface comprises at least one of an electrically-conducting polymer, an electrically-conducting foam, an electrically-conducting gel, an electrically-conducting fabric, and a metal.

5. The sensor of claim 4, wherein the electrically-conducting fabric comprises a plurality of electrically conducting threads, wires, or an electrically-conducting polymer embedded therein.

6. The sensor of claim 1, wherein the electrically-conducting interface is configured to be activated by a mechanical pressure at or above a predetermined threshold.

7. The sensor of claim 1, further comprising at least one of an amplifier, an analog to digital converter, and an energy source providing power to the sensor.

8. The sensor of claim 7, wherein the energy source is a battery, a rechargeable battery or a capacitor.

9. The sensor of claim 1, wherein the sensor is integrated in a conductive fabric comprising a plurality of electrically conducting threads or wires embedded therein, the plurality of conductive nodes are integrated within the conductive fabric, the conductive fabric being a bed sheet, a crib sheet, a blanket, a pillow case, or an item of clothing.

10. A sensor array for detecting physiological signals in a person comprising:
a plurality of conductive nodes each configured to be placed in contact with a person to detect an electrophysiological signal or biomechanical data from the person; and
a conductive fabric comprising a plurality of electrically conducting threads or wires embedded therein, the plurality of conductive nodes are integrated within the conductive fabric,
wherein each of the conductive nodes is electrically isolated from other conductive nodes.

11. The sensor array of claim 10, wherein the electrophysiological signal is selected from the group consisting of electroencephalography (EEG), electrocardiogram (EKG or ECG), electromyogram (EMG), and electrooculogram (EOG).

12. The sensor array of claim 10, wherein the biomechanical data corresponds to breathing or respiration of the person.

13. The sensor array of claim 10, wherein at least one of the plurality of conductive nodes is configured to be activated by a mechanical pressure at or above a predetermined threshold.

14. The sensor array of claim 10, wherein the plurality of conductive nodes are co-planar with a surface of the conductive fabric.

15. The sensor array of claim 10, wherein each of the conductive nodes forms a bump rising out of a surface of the conductive fabric.

16. The sensor array of claim 10, wherein the conductive fabric is a quilted meshwork and each of the plurality of conductive nodes is an electrically independent and conductive surface within the quilted meshwork.

17. The sensor array of claim 10, wherein the conductive fabric is a bed sheet, a crib sheet, a blanket, a pillow case, or an item of clothing.

18. The sensor array of claim 10, further comprising one or more energy sources, wherein the one or more energy sources are configured to provide a common float so as to short grounds of the one or more energy sources.

19. The sensor array of claim 10, further comprising one or more energy sources, wherein the one or more energy sources are configured to include at least two electrically-conducting interfaces, wherein at least one of the electrically-conducting interfaces provides at least one of a reference voltage and a ground.

20. The sensor array of claim 10, wherein at least one of the plurality of conductive nodes are configured to receive timing signals from a common clock or a master clock or a common timing signal intrinsic to the person.

21-37. (canceled)
